(19) United States
(12) Patent Application Publication
(10) Pub. No.: US 2012/0231970 A1
(43) Pub. Date: Sep. 13, 2012

(54) COLON CANCER MARKER AND METHOD FOR TESTING FOR COLON CANCER

(75) Inventors: Hitoshi Nakagama, Tokyo (JP); Naoto Tsuchiya, Tokyo (JP); Masashi Izumiya, Tokyo (JP)

(73) Assignee: JAPAN HEALTH SCIENCES FOUNDATION, Tokyo (JP)

(21) Appl. No.: 13/499,079

(22) PCT Filed: Sep. 30, 2010
(86) PCT No.: PCT/JP2010/067081

§ 371 Date: May 18, 2012

(30) Foreign Application Priority Data
Sep. 30, 2009 (JP) 2009-228029

(51) Int. Cl. C40B 30/04; C40B 40/06; C07H 21/02; G01N 21/64

(52) U.S. Cl. 506/9; 435/6.11; 506/16; 536/23.5

(57) ABSTRACT

Provided are a micro RNA which can be used as a colon cancer marker, a method for testing for colon cancer which uses the micro RNA which can be used as a colon cancer marker, and a test kit which can be used for the testing method. The colon cancer marker includes micro RNA molecules which are represented by any of the sequences 1 through 25. The method for testing for colon cancer in a subject involves preparing a sample, which contains a micro RNA molecule derived from the subject's colon tissue or colon cell, and detecting a micro RNA molecule represented by any of the sequences 1 through 25 present in the obtained sample.

### CROSS REFERENCE TO RELATED APPLICATIONS

This application claims priority to and benefit from Japanese patent application No. 2009-228029 filed on Sep. 30, 2009, the disclosure of which is incorporated herein by reference in its entirety.

### TECHNICAL FIELD

The present invention relates to a colon cancer marker comprising a specific microRNA. Furthermore, the present invention relates to a method for testing colon cancer using the microRNA as a marker.

### BACKGROUND ART

Although microRNA is an intracellular regulator of gene expression, and certain microRNA species are known to be extracellularly released in a form enveloped in a lipid membrane referred to as exosome. In addition, it is known that extracellular secretion of exosomes generally increases in cancer cells as compared to normal cells (Non-patent Literatures 1 and 2). It is also known that a microRNA that is specifically secreted from cancer cells can be a diagnosis marker (Non-patent Literatures 3 and 4).

Non-patent Literature 1 describes a microRNA that can be a diagnosis marker for ovarian cancer. Non-patent Literature 2 describes a microRNA that can be a diagnosis marker for lung cancer. Non-patent Literature 3 describes a microRNA that can be a diagnosis marker for colon cancer. Non-patent Literature 4 describes that a microRNA is detectable from vesicles in the peripheral blood.

### Citation List

### Patent Literature

Patent Literature 1: Japanese Patent Application Laid-Open (JP-A) No. 2009-124957

### Non-Patent Literatures

Non-patent Literature 1: TAYLOR, Douglas D. and GERCEL-TAYLOR, Cicek; MicroRNA signatures of tumor-derived exosomes as diagnostic biomarkers of ovarian cancer, Gynecologic Oncology, 2008, Vol. 110, pp.13-21.

Non-patent Literature 2: RABINOWITS, Guilherme; GERCEL-TAYLOR, Cicek; DAY, Jamie M.; TAYLOR, Douglas D.; and KLOECKER, Goetz H.; Exosomal MicroRNA: A Diagnostic Marker for Lung Cancer, Clinical Lung Cancer, 2009, Vol. 10, No. 1, pp. 42-46.

Non-patent Literature 3: NG, EK; CHONG, Wilson W S; SIN, Hongchuan; LAM, Emily K Y; SHIN, Vivian Y; YU, Jun; POON, Terence C W; NG, Simon S M; and SUNG, Joseph J Y; Differential expression of microRNAs in plasma of colorectal cancer patients: A potential marker for colorectal cancer screening.

Non-patent Literature 4: HUNTER, Melissa Piper et al., Detection of microRNA Expression in Human Peripheral Blood Microvesicles, PLOSONE November 2008, Vol. 3, Issue 11, e3694.

Non-patent Literature 5: Hadi Valadi, et al., Exosome-mediated transfer of mRNAs and microRNAs is a novel mechanism of genetic exchange between cells, Nature Cell Biology, June 2007, Vol. 9, No. 6, pp. 654-659.

The disclosures of Patent Literature 1 and Non-patent Literatures 1 to 5 are incorporated herein by reference in its entirety.

### DISCLOSURE OF THE INVENTION

### Problems to be Solved by the Invention

However, a specific microRNA that can be a marker for diagnosis of colon cancer has not been known as yet. Thus, an object of the present invention is to provide a microRNA that can be a colon cancer marker. Furthermore, an object of the present invention is to provide a method for testing colon cancer using this microRNA that can be a marker, and to provide a testing kit that can be used in this testing method.

### Means for Solving the Problems

The present inventors investigated to provide a microRNA that can be a colon cancer marker, and as results, found that 25 different microRNA species can be used as a colon cancer marker, which provides a method for testing colon cancer and a testing kit, and therefore completed the invention.

The present invention includes the following species:

[1] A colon cancer marker consisiting of a microRNA species represented by any one of Sequence Nos. 1 to 25.

[2] A colon cancer marker consisiting of a microRNA species represented by any one of Sequence Nos. 1, 2, 4, 5, 6, 8, 9, 11, 12, 14, 24, and 25.

[3] A method for testing for colon cancer in a subject, comprising steps of preparing a sample containing microRNA derived from colon tissue or colon cells of the subject, and detecting a microRNA species represented by any one of Sequence Nos. 1 to 25 that is present in the resulting sample.

[4] The method as described in [3], wherein the step of detecting a microRNA species is performed by bringing the sample into contact with a DNA-immobilized surface of a DNA array, onto which DNA fragments are immobilized, with the respective DNA fragments having a sequence that is complementary to at least a portion of at least one microRNA species represented by any one of Sequence Nos. 1 to 25, whereby presence of a microRNA species that hybridizes to the immobilized DNA fragments is detected in the sample.

[5] The method as described in [3], wherein the step of detecting a microRNA species comprises a step of performing reverse transcription reaction using at least one microRNA species represented by any one of Sequence Nos. 1 to 25 as a template to synthesize DNA fragments, a step of amplifying the resulting DNA fragments, and a step of detecting the amplified DNA fragments.

[6] The method as described in any one of [3] to [5], which uses at least one microRNA species represented by any one of Sequence Nos. 1, 2, 4, 5, 6, 8, 9, 11, 12, 14, 24, and 25.

[7] A DNA array comprising DNA fragments immobilized thereon, wherein the respective DNA fragments have a sequence that is complementary to at least a portion of at least one microRNA species represented by any one of Sequence Nos. 1 to 25.

[8] The DNA array as described in [7], wherein the respective immobilized DNA fragments have a sequence that is complementary to at least a portion of at least one microRNA species represented by any one of Sequence Nos. 1, 2, 4, 5, 6, 8, 9, 11, 12, 14, 24, and 25.

[9] A testing kit for colon cancer, comprising the DNA array as described in [7] or [8], and reagents for preparing a sample containing microRNA from colon tissue or colon cells of a subject.

### Effects of the Invention

According to the present invention, it is possible to provide a microRNA that can be a colon cancer marker. Furthermore, the present invention relates to a method and a kit for testing for colon cancer using the microRNA as a marker.

### BRIEF DESCRIPTION OF THE DRAWINGS

FIG. 1 shows microRNA profiles of the exosome fractions of the colon cancer cell lines and the normal colon cell line, which were obtained in Example 1.

FIG. 2 shows distribution of the normalized signal intensities, which were obtained in Example 2.

FIG. 3 shows distribution of the normalized signal intensities, which were obtained in Example 3.

### BEST MODES FOR CARRYING OUT THE INVENTION

### [Colon Cancer Marker]

The present invention relates to a colon cancer marker comprising a microRNA species represented by any one of Sequence Nos. 1 to 25 shown below.

| (Sequence No. 1) | |
|---|---|
| miR-638: | AGGGAUCGCGGGCGGGUGGCGGCCU |
| (Sequence No. 2) | |
| miR-1915: | CCCCAGGGCGACGCGGCGGG |
| (Sequence No. 3) | |
| miR-630: | AGUAUUCUGUACCAGGGAAGGU |
| (Sequence No. 4) | |
| miR-1268: | CGGGCGUGGUGGUGGGGG |
| (Sequence No. 5) | |
| miR-1207-5p: | UGGCAGGGAGGCUGGGAGGGG |
| (Sequence No. 6) | |
| miR-572: | GUCCGCUCGGCGGUGGCCCA |
| (Sequence No. 7) | |
| miR-1246: | AAUGGAUUUUUGGAGCAGG |
| (Sequence No. 8) | |
| miR-483-5p: | AAGACGGGAGGAAAGAAGGGAG |
| (Sequence No. 9) | |
| miR-1225-5p: | GUGGGUACGGCCCAGUGGGGGG |
| (Sequence No. 10) | |
| miR-575: | GAGCCAGUUGGACAGGAGC |
| (Sequence No. 11) | |
| miR-1202: | GUGCCAGCUGCAGUGGGGGAG |
| (Sequence No. 12) | |
| miR-765: | UGGAGGAGAAGGAAGGUGAUG |
| (Sequence No. 13) | |
| miR-320c: | AAAAGCUGGGUUGAGAGGGU |
| (Sequence No. 14) | |
| miR-513a-5p: | UUCACAGGGAGGUGUCAU |
| (Sequence No. 15) | |
| miR-494: | UGAAACAUACACGGGAAACCUC |
| (Sequence No. 16) | |
| miR-939: | UGGGGAGCUGAGGCUCUGGGGGUG |
| (Sequence No. 17) | |
| miR-1290: | UGGAUUUUUGGAUCAGGGA |
| (Sequence No. 18) | |
| miR-1275: | GUGGGGGAGAGGCUGUC |
| (Sequence No. 19) | |
| miR-671-5p: | AGGAAGCCCUGGAGGGGCUGGAG |
| (Sequence No. 20) | |
| miR-223: | UGUCAGUUUGUCAAAUACCCCA |
| (Sequence No. 21) | |
| miR-25: | CAUUGCACUUGUCUCGGUCUGA |
| (Sequence No. 22) | |
| miR-92a: | UAUUGCACUUGUCCCGGCCUGU |
| (Sequence No. 23) | |
| miR-1183: | CACUGUAGGUGAUGGUGAGAGUGGGCA |
| (Sequence No. 24) | |
| miR-1224-5p: | GUGAGGACUCGGGAGGUGG |
| (Sequence No. 25) | |
| miR-188-5p: | CAUCCCUUGCAUGGUGGAGGG |

In order to isolate a marker that allows early diagnosis of human colon cancer, a microRNA that is specifically secreted from colon cancer cells, was identified. A specific method is outlined as follows, which will be described in detail in Example 1. Using 5 different colon cancer cell lines and a normal colon cell line (FHC), exosomes from culture supernatants of the respective cell lines were concentrated in accordance with an ordinary method (Non-patent Literature 5) to obtain exosome fractions. From the respective fractions, RNAs were isolated, and microRNAs concentrated in the exosome fractions were exhaustively analyzed using a microRNA microarray manufactured by Agilent Technologies, Inc., and their profiles were obtained.

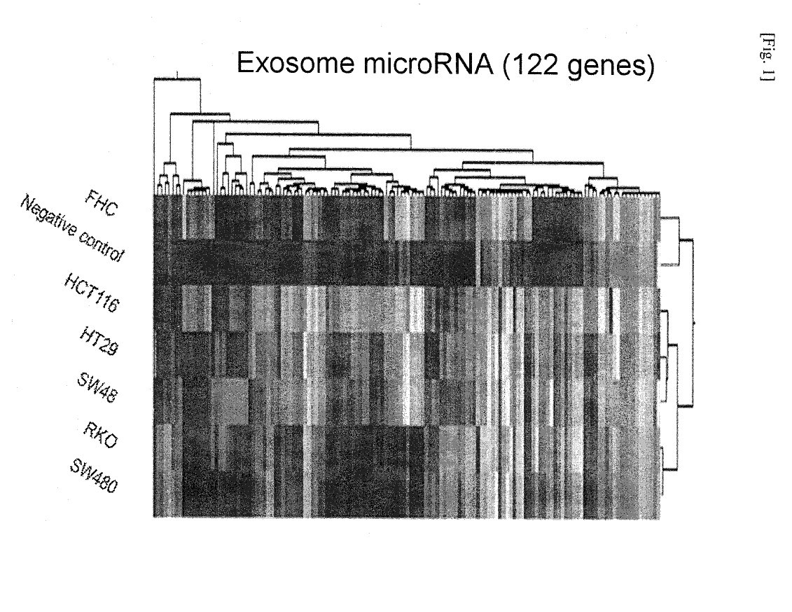

Based on the results, it was determined that profiles of microRNAs of the exosome fractions of the colon cancer cells were remarkably different from those of the normal colon cells. That is to say, it is shown that microRNAs extracellularly secreted are greatly different between normal cells and cancer cells (FIG. 1).

Furthermore, it was also found that there is high similarity in the profiles of secreted microRNAs among the colon cancer cell lines. Using these profiles, were selected microRNAs that were commonly secreted from the 5 colon cancer cell lines, but was scarcely secreted from the normal cells. In Table 1, with respect to the 24 microRNA species, the average signal intensities of the respective microRNAs isolated from the five colon cancer cell lines are shown, respectively, as a ratio to the corresponding signal intensity from the normal cell line (FHC).

As shown in Table 1, it was found that the 24 different microRNAs were secreted from all the 5 cancer cell lines. These included microRNAs such as miR-572 and 1225-5p, with their secretion increased by 50-fold or more in the cancer cells in comparison to the normal cells. In addition, even for miR-765, which was the lowest, the secretion in the cancer cells increased by 3.6-fold in comparison to the normal cells. Consequently, these 24 microRNA species can be applied as a marker that can be used in diagnosis of colon cancer. In view that the degree of an increase in secretion is high in cancer cells, a microRNA species that increases by 10-fold or more in comparison to normal cells is preferable as a marker that is used in diagnosis of colon cancer, a microRNA species that increases by 20-fold or more in comparison to normal cells is more preferable as a marker that is used in diagnosis of colon cancer, and a microRNA species that increases by 30-fold or more in comparison to a normal cell is further preferable as a marker that is used in diagnosis of colon cancer.

TABLE 1
Candidates of microRNAs specifically secreted from 5 colon cancer
cell lines

| | Signal Intensity (Average value of | Fold increase |
|---|---|---|
| MicroRNA | 5 colon cancer cell lines) | (/FHC) |
| hsa-miR-638 | 930.0 | 38.0 |
| hsa-miR-1915 | 752.7 | 41.2 |
| hsa-miR-630 | 385.9 | 16.9 |
| hsa-miR-1268 | 139.5 | 8.1 |
| hsa-miR-1207-5p | 108.9 | 7.6 |
| hsa-miR-572 | 100.6 | 100.6 |
| hsa-miR-1246 | 99.4 | 5.9 |
| hsa-miR-483-5p | 84.4 | 9.2 |
| hsa-miR-1225-5p | 62.7 | 62.7 |
| hsa-miR-575 | 57.7 | 57.7 |
| hsa-miR-1202 | 47.4 | 8.0 |
| hsa-miR-765 | 37.5 | 3.6 |
| hsa-miR-320c | 32.4 | 32.4 |
| hsa-miR-513a-5p | 29.1 | 29.1 |
| hsa-miR-494 | 26.3 | 26.3 |
| hsa-miR-939 | 25.5 | 4.5 |
| hsa-miR-1290 | 23.9 | 23.9 |
| hsa-miR-1275 | 23.8 | 23.8 |
| hsa-miR-671-5p | 14.3 | 14.3 |
| hsa-miR-223 | 10.5 | 10.5 |
| hsa-miR-25 | 9.5 | 9.5 |
| hsa-miR-92a | 9.3 | 9.3 |
| hsa-miR-1183 | 8.5 | 8.5 |
| hsa-miR-1224-5p | 8.4 | 8.4 |

As described above, the 24 different microRNAs shown in Table 1 are represented in the sequence list as Sequence Nos. 1 to 24 starting in order from top to bottom.

The 24 different microRNAs are specifically secreted from colon cancer cell lines, and may be possibly applied to serum diagnosis of cancer. Among them, a microRNA secreted from colon cancer cells at an early stage can be used as a marker for early diagnosis of colon cancer.

Furthermore, as shown in Examples 2 and 3, from the results of plasma exosomal microRNA comparison conducted on the exosomes collected from blood samples of healthy controls and colon cancer patients, it was determined that 11 different microRNAs represented by any one of Sequence Nos. 1, 2, 4, 5, 6, 8, 9, 11, 12, 14, and 24 can be applied to serum diagnosis of cancer in very great likelihood among the microRNAs shown in Table 1. Furthermore, from the results of plasma exosomal microRNA comparison, it was determined that the microRNA referred to as miR-188-5p (Sequence No. 25), though not listed in Table 1, could be applied to serum diagnosis of cancer in very great likelihood, similarly as the 11 different microRNAs.

### [Method for Testing for Colon Cancer]

The present invention encompasses a method for testing colon cancer using the marker of the present invention. The method for testing colon cancer of the present invention is a method using at least one microRNA species represented by any one of Sequence Nos. 1 to 25. The microRNA species of the present invention can be a marker, and various methods that can implement the microRNA species as a marker may be adopted. Examples of the method include (a) a method using a DNA array on which DNA fragments are immobilized, wherein the respective DNA fragments have a sequence that is complementary to at least a portion of at least one kind of microRNA species represented by any one of Sequence Nos. 1 to 25, (b) a method comprising a process of performing reverse transcription reaction using at least one microRNA species represented by any one of Sequence Nos. 1 to 25 as a template to synthesize DNA fragments, a process of amplifying the resulting DNA fragments, and a process of detecting the amplified DNA fragments, (c) a detection method of high sensitivity using a solid-phased bead, or a carrier that is similar to the bead, in which synthetic nucleic acids having a sequence that is complementary to at least a portion of at least one microRNA species represented by any one of Sequence Nos. 1 to 25, are loaded, and the like.

In any one of the methods (a) to (c), a sample containing microRNA presumably derived from colon tissue or colon cells of a subject is prepared. The microRNA that is derived from the colon tissue or colon cells of a subject may be suitably prepared from, for example, the body fluid (the serum and the like), the urine, the stool, and the like of a patient.

A DNA array is used in the method (a). First, the DNA array will be described.

The DNA array used in the present invention comprises DNA fragments that are immobilized on the said array, with each DNA fragment having a sequence that is complementary to at least a portion of at least one microRNA species represented by any one of Sequence Nos. 1 to 25. The DNA array used in the present invention may comprise DNA fragments having sequences complementary to all the respective 25 different microRNA species represented by Sequence Nos. 1 to 25 immobilized thereon, or may comprise DNA fragments having sequences complementary, respectively, to a few (1 or 2 or more) of the 25 different microRNA species immobilized thereon. In particular, as for the DNA fragment having a sequence that is complementary to at least a portion of a microRNA species, a DNA fragment having a sequence that is complementary to, for example, a sequence of 10 to 20 bases of a microRNA species is suitable, in view of obtaining assured and specific hybridization with the target microRNA species. However, it is not intended that the number of nucleotide bases be limited to the range of 10 to 20. Furthermore, the DNA strand is suitably labeled for detecting hybridization of the target microRNA species. For example, either one of the target microRNA species and the immobilized DNA may be labeled with Cy3, and the other may be labeled with Cy5. The labeling with Cy3 and Cy5 may be performed with an ordinary method.

The present invention also encompasses the DNA array.

The sample obtained above is brought into contact with the DNA-immobilized surface of the DNA array. This contact may be implemented similarly to a conventional way to make a contact between a sample comprising microRNA and a DNA array. For example, (i) microRNAs labeled with Cy3 and derived from a patient subject are added onto a DNA array, and incubated at a temperature optimal for hybridization. (ii) A mixture of microRNAs derived from a patient subject and labeled with Cy3, and a sample derived from a healthy control and labeled with Cy5 is added onto and brought in contact with the DNA array, whereby the sequence of each microRNA specific to the sample of the patient can be determined.

After the contact, if a microRNA species that hybridizes to the immobilized DNA is present or not in the sample, can be detected using, for example, the labels to the microRNA species and the immobilized DNA. When the label is, for example, a fluorophore, the presence or absence of fluorescence or the color of fluorescence can be also detected. In addition, for a sample labeled with two different fluorophores, the ratio of the fluorescence intensities can be detected.

In the method (b), the detection of the microRNA species is performed with a method that comprises (i) a process of performing reverse transcription reaction using at least one microRNA species represented by any one of Sequence Nos. 1 to 25 as a template to synthesize DNA fragments, (ii) a process of amplifying the resulting DNA fragments, and (iii) a process of detecting the amplified DNA fragments.

(i) Reverse Transcription Reaction Using Microrna Species as Template

The reverse transcription reaction using the microRNA species as a template may be implemented in the presence of a reverse transcription enzyme and nucleotides that are a substrate for the enzyme, and further a primer. The reverse transcription reaction may be performed under a condition applicable to all the 25 different microRNA species represented by Sequence Nos. 1 to 25 to be used as the template, or under a condition applicable to a few (1 or 2 or more) of the 25 different microRNA species as the template.

(ii) Process of Amplifying Resulting DNA Fragments

The process of amplifying the resulting DNA fragments may be performed using, for example, a PCR method. The PCR method may be also performed under a condition applicable to the reverse transcripts from all the 25 different microRNA species to be used as the template, or may be performed under a condition applicable to a few (1 or 2 or more) of the 25 different microRNA species to be used as the template.

(iii) Process of Detecting Amplified DNA Fragments

The amplified DNA fragments may be suitably detected by the fluorescent label that had been introduced on some nucleotides used in the amplification reaction of the DNA fragments. In addition, a probe capable of detecting a specific amplified product by fluorescence (for example, TaqManprobe manufactured by Applied Biosystems), and the like may be also used.

In comparison to the method using a DNA array, the method (b) can give higher sensitivity depending on the degree of amplification since the method (b) comprises a process of amplifying DNA fragments. For example, an increase in the detection sensitivity by about 10-fold of the microarray also allows serum diagnosis of cancer at an early stage.

The method (c) can be carried out as described below. The synthetic nucleic acid having a sequence that is complementary to at least a part of at least one microRNA species represented by any one of Sequence Nos. 1 to 25 suitably has a sequence that is complementary to, for example, a sequence of 10 to 20 bases of a DNA fragment, in view that it can hybridize surely and specifically to the target microRNA species. However, it is not intended that the number of bases be limited to the range of 10 to 20. The synthetic nucleic acid can be loaded onto a carrier such as solid-phase resin beads with an ordinary method. Furthermore, the synthetic nucleic acid suitably has a label for detecting hybridization of the target microRNA species. For example, either one of the target microRNA molecule and the immobilized synthetic nucleic acid may be labeled with Cy3, and the other may be labeled with Cy5. Labeling with Cy3 and Cy5 may be performed with an ordinary method. The resulting sample containing microRNA is brought into contact with the solid-phase beads loaded with the synthetic nucleic acid, and then if the microRNA species that hybridizes to the immobilized synthetic nucleic acid is present or not in the sample can be detected by, for example, labeling the synthetic nucleic acid and the immobilized DNA fragment. On one set of beads, for example, one microRNAspecies is fixed, and several different sets of such beads may be used in parallel. However, the method is not intended to be limited to such an embodiment.

The method for detecting microRNA may be also carried out by referring to the description of Patent Literature 1, in addition to the methods described in (a) to (c).

### [Testing Kit]

The present invention also encompasses a colon cancer testing kit that comprises the DNA array of the present invention, and reagents for preparing a sample containing microRNA derived from colon tissue or colon cells of a subject. Reagents for preparing a sample containing microRNA include, for example, a buffer for collecting RNAs in a blood sample, or a set of beads on which proteins A and G are immobilized for removing unnecessary antibodies and the like in the blood. Furthermore, examples of the reagents include an organic solvent, an acidic phenol, and the like for collecting RNA species. In addition, examples of the reagents include a resin-packed column for isolating and purifying RNA species by their sizes, and the like. In addition, examples of the reagents include a PCR primer set for detecting specific microRNAs (the 25 different microRNAs), and enzymes necessary for PCR reaction, a buffer, fluorescent-labeled nucleotides, and the like.

### EXAMPLES

Hereinafter, the present invention will be further described in details with Examples. However, Examples described below are illustrative, and it is not intended that the scope of the present invention is limited to these Examples.

### Example 1

<Identification of MicroRNA Secreted from Colon Cancer Cell Line>

MicroRNAs specifically secreted from colon cancer cells were identified in order to isolate a marker of early diagnosis for human colon cancer.

(Method)

Cell Lines

Human fetal colon-derived cell line, FHC (normal cell line) and 5 different colon cancer cell lines (HCT 116, HT-29, RKO, SW48, and SW480) were used.

Isolation of Exosomes

Each cell line was cultured for 48 hours, and the culture medium (culture supernatant) was collected. The culture supernatant was centrifuged at 500×g for 5 minutes, and the supernatant was collected. The supernatant was further centrifuged at a high speed (16,500×g) and the insoluble fractions were removed, and then filtered with a 0.2 μm filter. The filtrate was ultra-centrifuged at 120,000×g, and the exosome fraction was isolated.

Preparation of RNA

Total RNA contained in the exosome fraction was precipitated using Trizol-LS (Invitrogen). The quality of the RNA was confirmed with Agilent 2100 Bioanalyzer.

Analysis of MicroRNA Microarray

100 ng of the precipitated total RNA was labeled as a template with Cy3. Detection of microRNA species was performed using a microRNA microarray manufactured by Agilent. This array is loaded with oligo probes that specifically detect 851 human microRNAs. Numerical conversion and statistical analysis of the signal values were performed using GeneSpring GX10 software.

The results are shown in FIG. 1. The negative control is a medium comprising 10% bovine fetal serum (medium containing no culture of cells). It was determined that the profiles of microRNAs contained in the exosome fractions of the colon cancer cells were remarkably different from those of the normal colon cells.

Furthermore, it was found that the 24 different microRNAs shown in Table 1 were secreted from all the cancer cell lines.

### Example 2

Exosomes were collected using an ultra-centrifuge method from the blood samples (1 mL plasma sample each kept at −20° C.) of 14 healthy controls and 10 colon cancer (colorectal cancer) patients (40 to 60 years old: see Table 2) with no prior chemotherapy. RNAs were extracted from the total volume of the collected exosomes, and a portion adjusted to 10 was taken as a sample for Agilent oligonucleotide microarray/human microRNA V3 microarray (loaded with 851 microRNAs). The resulting array data (signal intensity) was divided with the amount of RNA (ng), and the calculated value was taken as the normalized signal intensity (AU).

TABLE 2
Profiles of healthy controls (n = 14) and CRC patients (n = 10)

| Healthy control | | | CRC patient | | |
|---|---|---|---|---|---|
| ID | Gender | Age | ID | Gender | Age |
| H1 | M | 47 | CRC1 | M | 50 |
| H2 | M | 41 | CRC2 | F | 46 |
| H3 | F | 51 | CRC3 | F | 52 |
| H4 | F | 41 | CRC4 | M | 59 |
| H5 | M | 48 | CRC5 | M | 52 |
| H6 | M | 54 | CRC6 | M | 60 |
| H7 | M | 42 | CRC7 | M | 48 |
| H8 | M | 47 | CRC8 | M | 58 |
| H9 | M | 52 | CRC9 | M | 45 |
| H10 | F | 52 | CRC10 | F | 59 |
| H11 | F | 52 | | | |
| H12 | M | 58 | | | |
| H13 | M | 43 | | | |
| H14 | M | 50 | | | |
| Average | | 48.4 | Average | | 52.9 |

Among 31 different microRNAs selected from the experimental results of the culture supernatant-derived exosomes of human normal colon-derived FHC cell line and 5 different colon cancer cell lines (HCT116, HT29, RKO, SW48, and SW480), 14 different microRNAs that met the two conditions described below were selected (see Table 3).
Detected in 70% (7 persons) or more of the colon cancer patients
Significantly higher in comparison to the data of healthy controls (Student's t-test two-tailed assay p<0.05)

TABLE 3
Plasma exosomal microRNAs compared between healthy controls
(n = 14) and CRC patients (n = 10)

| | Healthy control | CRC patient | TTEST* |
|---|---|---|---|
| | Average | Average | p = ** |
| number of exosomal | 82.5 | 92.6 | 0.2585 |
| microRNA species | | | |
| Exosomal RNA (ng)/ | 3.39 | 3.19 | 0.7101 |
| 1 mL plasma | | | |

| Signal | | | | | |
|---|---|---|---|---|---|
| intensity/ng | | | | | |
| exosomal RNA | Average | Detected*** | Average | Detected*** | |
| miR-1202 | 96.3 | (14) | 305.7 | (10) | 0.0001 |
| miR-1225-5p | 61.8 | (14) | 216.6 | (10) | 0.0110 |
| miR-638 | 50.9 | (14) | 134.7 | (10) | 0.0232 |
| miR-1915 | 28.7 | (14) | 93.4 | (10) | 0.0097 |
| miR-1207-5p | 9.8 | (14) | 86.6 | (10) | 0.0459 |
| miR-1224-5p | 1.4 | (0) | 4.7 | (7) | 0.0072 |
| miR-1268 | 16.0 | (14) | 40.9 | (10) | 0.0021 |
| miR-188-5p | 16.6 | (14) | 35.4 | (10) | 0.0005 |
| miR-483-5p | 6.3 | (13) | 21.9 | (10) | 0.0155 |
| miR-572 | 4.5 | (9) | 14.8 | (9) | 0.0160 |
| miR-939 | 2.0 | (5) | 12.7 | (10) | 0.0000 |
| miR-1290 | 4.9 | (9) | 11.7 | (10) | 0.0126 |
| miR-765 | 2.8 | (9) | 9.6 | (9) | 0.0255 |
| miR-513a-5p | 1.7 | (2) | 3.3 | (8) | 0.0103 |

*Student's t-test
**two-tailed p-value
***number of samples detected by microRNA microarray assay for each microRNA species

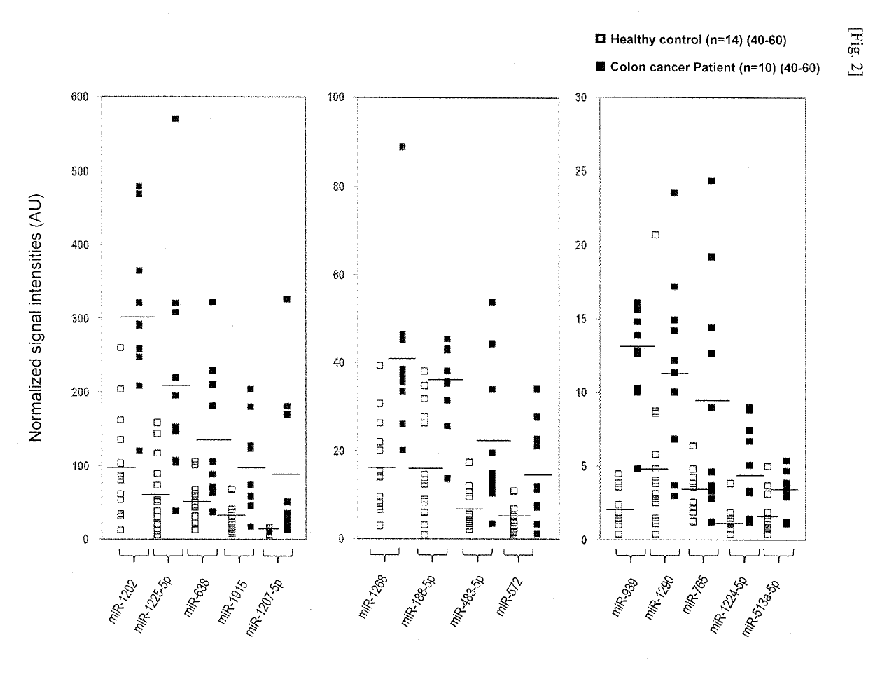

FIG. 2 shows the normalized signal intensities.

Bule box: Healthy control

Red box: Colon cancer Patient

Bar: Average Value

Vertical Axis: Normalized signal intensity (AU)

Horizontal Axis: microRNA species: listed in descending order, from left, of the results (average values) in the colon cancer patients.

### Example 3

Exosomes were collected using an ultra-centrifuge method from blood samples (1 mL plasma sample kept at −20 degree) of 20 healthy controls and 21 colon cancer (colorectal cancer) patients (27 to 78 years old: see Table 4) with no prior chemotherapy. The subsequent procedures were conducted similarly to those described in FIG. 2. Condition 1 was satisfied by a RNA species detected in 14 persons or more (see Table 5).

1. Detected in 70% (7 persons) or more of the colon cancer patients

2. Significantly higher in comparison to the data of healthy controls (Student's t-test two-tailed assay p<0.05)

TABLE 4
Profiles of healthy controls (n = 20) and CRC patients (n = 21)

| Healthy control | | | CRC patient | | |
|---|---|---|---|---|---|
| ID | Gender | Age | ID | Gender | Age |
| H1 | M | 47 | CRC1 | M | 50 |
| H2 | M | 41 | CRC2 | F | 46 |
| H3 | F | 51 | CRC3 | F | 52 |
| H4 | F | 41 | CRC4 | M | 59 |
| H5 | M | 48 | CRC5 | M | 52 |
| H6 | M | 54 | CRC6 | M | 60 |
| H7 | M | 42 | CRC7 | M | 48 |
| H8 | M | 47 | CRC8 | M | 58 |
| H9 | M | 52 | CRC9 | M | 45 |
| H10 | F | 52 | CRC10 | F | 59 |
| H11 | F | 52 | CRC11 | F | 62 |
| H12 | M | 58 | CRC12 | F | 62 |
| H13 | M | 43 | CRC13 | F | 61 |
| H14 | M | 50 | CRC14 | F | 66 |
| H15 | F | 35 | CRC15 | M | 67 |
| H16 | M | 37 | CRC16 | M | 78 |
| H17 | M | 38 | CRC17 | F | 63 |
| H18 | F | 38 | CRC18 | F | 64 |
| H19 | M | 27 | CRC19 | M | 70 |
| H20 | F | 29 | CRC20 | F | 61 |
| | | | CRC21 | M | 67 |
| Average | | 44.1 | Average | | 59.5 |

TABLE 5
Plasma exosomal microRNAs compared between healthy controls
(n = 20) and CRC patients (n = 21)

| | Healthy control | CRC patient | TTEST* |
|---|---|---|---|
| | Average | Average | p = ** |
| number of exosomal | 81.7 | 89.2 | 0.2577 |
| microRNA species | | | |
| Exosomal RNA (ng)/ | 3.94 | 3.64 | 0.4978 |
| 1 mL plasma | | | |

| Signal | | | | | |
|---|---|---|---|---|---|
| intensity/ng | | | | | |
| exosomal RNA | Average | Detected*** | Average | Detected*** | |
| miR-1202 | 90.9 | (20) | 252 | (21) | 0.0001 |
| miR-1225-5p | 58.5 | (20) | 180.9 | (21) | 0.0036 |
| miR-638 | 50.1 | (20) | 125.4 | (21) | 0.0007 |
| miR-1915 | 26.4 | (20) | 89.4 | (21) | 0.0001 |
| miR-1207-5p | 9.2 | (19) | 81.4 | (21) | 0.0059 |
| miR-1224-5p | 1.3 | (0) | 4.9 | (14) | 0.0001 |
| miR-1268 | 15.4 | (20) | 32.6 | (21) | 0.0004 |
| miR-188-5p | 15.7 | (19) | 28.6 | (21) | 0.0017 |
| miR-483-5p | 7.5 | (19) | 22.3 | (21) | 0.0040 |
| miR-572 | 4.2 | (12) | 13.9 | (20) | 0.0002 |
| miR-939 | 2.1 | (8) | 11.7 | (21) | 0.0000 |
| miR-1290 | 4.8 | (14) | 9.2 | (19) | 0.0164 |
| miR-765 | 4.0 | (14) | 8.6 | (18) | 0.0378 |
| miR-513a-5p | 1.7 | (3) | 2.7 | (15) | 0.0138 |

*Student's t-test
**two-tailed p-value
***number of samples detected by microRNA microarray assay for each microRNA species

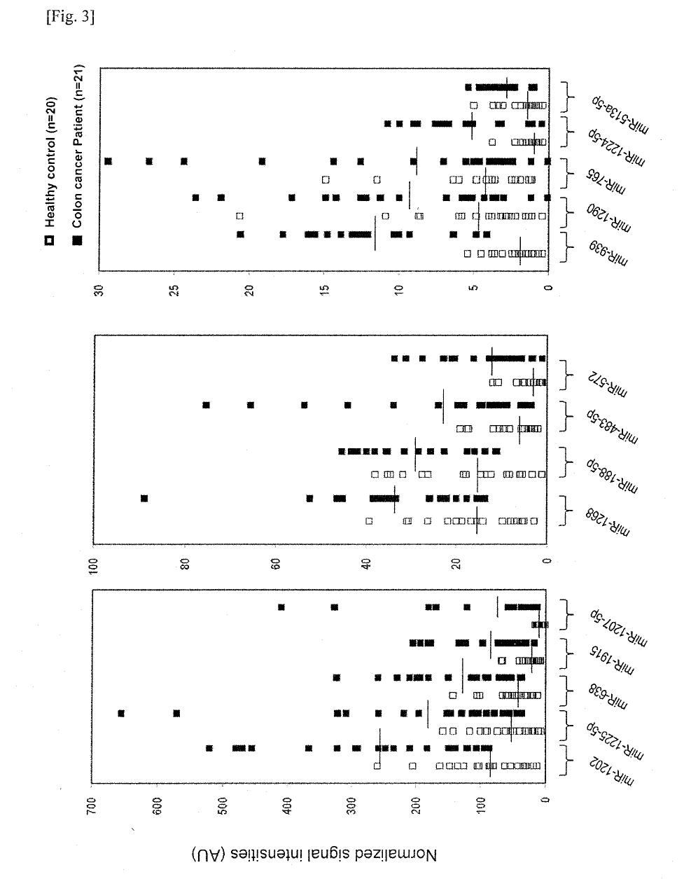

FIG. 3 shows the normalized signal intensities.

Blue box: Healthy control

Red box: Colon cancer Patient

Bar: Average Value

Vertical Axis: Normalized signal intensity (AU)

Horizontal Axis: microRNA species: listed in descending order, from left, of the results (average values) in the colon cancer patients.

From the results shown in FIGS. 2 and 3, the same microRNAs happened to be selected although there were some differences in the average value and in the p value. From the results shown in FIGS. 2 and 3, the 12 different microRNAs, i.e., 11 different microRNAs represented by Sequence Nos. 1, 2, 4, 5, 6, 8, 9, 11, 12, 14, and 24 among the 24 microRNA species shown in Table 1, and miR-188-5p (not listed in Table 1) (Sequence No. 25) can be all used in serum diagnosis of cancer in very great likelihood, and can be also used as a marker for early diagnosis of colon cancer.

### INDUSTRIAL APPLICABILITY

The present invention is useful in a field related to a method or a kit for testing for colon cancer.

1. A colon cancer marker consisting of a microRNA species represented by any one of Sequence Nos. 1 to 25.

2. A colon cancer marker consisting of a microRNA species represented by any one of Sequence Nos. 1, 2, 4, 5, 6, 8, 9, 11, 12, 14, 24 and 25.

3. A method for testing for colon cancer in a subject, comprising steps of preparing a sample containing microRNA derived from colon tissue or colon cells of the subject, and detecting a microRNA species represented by any one of Sequence Nos. 1 to 25 that is present in the resulting sample.

4. The method as described in claim 3, wherein the step of detecting a microRNA species is performed by bringing the sample into contact with a DNA-immobilized surface of a DNA array, onto which DNA fragments are immobilized, with each DNA fragment having a sequence that is complementary to at least a portion of at least one microRNA species represented by any one of Sequence Nos. 1 to 25, whereby presence of a microRNA species that hybridizes to the immobilized DNA fragments is detected in the sample.

5. The method as described in claim 3, wherein the step of detecting a microRNA species comprises a step of performing reverse transcription reaction using at least one microRNA species represented by any one of Sequence Nos. 1 to 25 as a template to synthesize DNA fragments, a step of amplifying the resulting DNA fragments, and a step of detecting the amplified DNA fragments.

6. The method as described in claim 3, which uses at least one microRNA species represented by any one of Sequence Nos. 1, 2, 4, 5, 6, 8, 9, 11, 12, 14, 24 and 25.

7. A DNA array comprising DNA fragments immobilized thereon, wherein the respective DNA fragments have a sequence that is complementary to at least a portion of at least one microRNA species represented by any one of Sequence Nos. 1 to 25.

8. The DNA array as described in claim 7, wherein the respective immobilized DNA fragments have a sequence that is complementary to at least a portion of at least one microRNA species represented by any one of Sequence Nos. 1, 2, 4, 5, 6, 8, 9, 11, 12, 14, 24 and 25.

9. A testing kit for colon cancer, comprising the DNA array as described in claim 7, and reagents for preparing a sample containing microRNA from colon tissue or colon cells of a subject.

10. The method as described in claim 4, which uses at least one microRNA species represented by any one of Sequence Nos. 1, 2, 4, 5, 6, 8, 9, 11, 12, 14, 24 and 25.

11. The method as described in claim 5, which uses at least one microRNA species represented by any one of Sequence Nos. 1, 2, 4, 5, 6, 8, 9, 11, 12, 14, 24 and 25.

12. A testing kit for colon cancer, comprising the DNA array as described in claim 8, and reagents for preparing a sample containing microRNA from colon tissue or colon cells of a subject.


SEQUENCE LISTING (25 sequences)

SEQ ID NO: 1 - RNA, 25 nt, Homo sapiens
agggaucgcgggcggguggcggccu

SEQ ID NO: 2 - RNA, 20 nt, Homo sapiens
ccccagggcgacgcggcggg

SEQ ID NO: 3 - RNA, 22 nt, Homo sapiens
aguauucuguaccagggaaggu

SEQ ID NO: 4 - RNA, 18 nt, Homo sapiens
cgggcguggugguggggg

SEQ ID NO: 5 - RNA, 21 nt, Homo sapiens
uggcagggaggcugggagggg

SEQ ID NO: 6 - RNA, 20 nt, Homo sapiens
guccgcucggcgguggccca

SEQ ID NO: 7 - RNA, 19 nt, Homo sapiens
aauggauuuuuggagcagg

SEQ ID NO: 8 - RNA, 22 nt, Homo sapiens
aagacgggaggaaagaagggag

SEQ ID NO: 9 - RNA, 22 nt, Homo sapiens
guggguacggcccagugggggg

SEQ ID NO: 10 - RNA, 19 nt, Homo sapiens
gagccaguuggacaggagc

SEQ ID NO: 11 - RNA, 21 nt, Homo sapiens
gugccagcugcagugggggag

SEQ ID NO: 12 - RNA, 21 nt, Homo sapiens
uggaggagaaggaaggugaug

SEQ ID NO: 13 - RNA, 20 nt, Homo sapiens
aaaagcuggguugagagggu

SEQ ID NO: 14 - RNA, 18 nt, Homo sapiens
uucacagggaggugucau

SEQ ID NO: 15 - RNA, 22 nt, Homo sapiens
ugaaacauacacgggaaaccuc

SEQ ID NO: 16 - RNA, 24 nt, Homo sapiens
uggggagcugaggcucugggggug

SEQ ID NO: 17 - RNA, 19 nt, Homo sapiens
uggauuuuuggaucaggga

SEQ ID NO: 18 - RNA, 17 nt, Homo sapiens
gugggggagaggcuguc

SEQ ID NO: 19 - RNA, 23 nt, Homo sapiens
aggaagcccuggaggggcuggag

SEQ ID NO: 20 - RNA, 22 nt, Homo sapiens
ugucaguuugucaaauacccca

SEQ ID NO: 21 - RNA, 22 nt, Homo sapiens
cauugcacuugucucggucuga

SEQ ID NO: 22 - RNA, 22 nt, Homo sapiens
uauugcacuugucccggccugu

SEQ ID NO: 23 - RNA, 27 nt, Homo sapiens
cacuguaggugauggugagagugggca

SEQ ID NO: 24 - RNA, 19 nt, Homo sapiens
gugaggacucgggaggugg

SEQ ID NO: 25 - RNA, 21 nt, Homo sapiens
caucccuugcaugguggaggg